(19) United States
(12) Patent Application Publication
(10) Pub. No.: US 2016/0324458 A1
(43) Pub. Date: Nov. 10, 2016

(54) Suppository Insertion Tool

(71) Applicant: James Doyle McCormick, Salt Lake City, UT (US)

(72) Inventor: James Doyle McCormick, Salt Lake City, UT (US)

(21) Appl. No.: 15/099,223

(22) Filed: Apr. 14, 2016

Related U.S. Application Data
(60) Provisional application No. 62/157,076, filed on May 5, 2015.

(51) Int. Cl. A61B 5/00; A61M 31/00

(52) U.S. Cl. CPC A61B 5/4255; A61M 31/007

(57) ABSTRACT

A suppository insertion tool is a device utilized to administer a suppository within the body as well as to detect the presence of an obstruction within the bowels. A semi-rigid plunger member is telescopically engaged into a tubular semi-rigid sleeve member. A suppository-receiving flexible end of the tubular semi-rigid sleeve member and an expandable opening traversing into the suppository-receiving flexible end are expanded when the semi-rigid plunger member is engaged into the tubular semi-rigid sleeve member. A suppository may then be inserted through the expandable opening. A plurality of color-coded blockage indicators may be utilized to detect the presence and severity of an obstruction within the bowels. The plurality of color-coded blockage indicators is offset from each other to accurately gauge the severity of an obstruction. A chemical reactant coating for various applications may be applied to a sleeve tubular body of the tubular semi-rigid sleeve member.

The current application claims a priority to the U.S. Provisional Patent application Ser. No. 62/157,076 filed on May 5, 2015.

### FIELD OF THE INVENTION

The present invention relates generally to a device for administering a suppository. More specifically, the present invention is a suppository insertion tool that is capable of detecting the presence of an obstruction within the bowels.

### BACKGROUND OF THE INVENTION

A suppository is a system for delivering a drug in which the suppository is inserted into a body orifice (most commonly the rectum) and dissolved before entering the bloodstream. Suppositories are commonly utilized to deliver drugs such as laxatives and hemorrhoid medications. Because suppositories are most often administered by hand, the process can be unsanitary, painful, and difficult. Suppositories must generally be administered deeply into the appropriate orifice in order to be effective. As such, improper administration by hand can altogether negate the effectiveness of a suppository.

The present invention is a suppository insertion tool that is utilized to administer a suppository. The present invention is additionally capable of detecting the presence of an obstruction within the bowels. When administering a suppository, the present invention is designed in a manner such that the suppository is inserted deeply into the appropriate body orifice before being delivered to the drug administration site. The present invention is designed to be convenient to handle and maneuver during the insertion of a suppository. The present invention is additionally utilized to detect the presence of an obstruction within the bowels.

### BRIEF DESCRIPTION OF THE DRAWINGS

FIG. 1 is an exploded perspective view of the present invention.

FIG. 2 is an exploded front view of the present invention.

FIG. 3 is a bottom view of the tubular semi-rigid sleeve member.

FIG. 4 is a front view of the present invention with the blockage detection sleeve.

FIG. 5 is a side view of the present invention with the chemical reactant coating.

FIG. 6 is a cross-sectional view taken alone line A-A of FIG. 5.

FIG. 7 is a detail view of the present invention taken from circle B of FIG. 6.

FIG. 8 is a side view of the present invention in the expanded configuration.

FIG. 9 is a cross-sectional view of the present invention taken along line C-C of FIG. 8.

FIG. 10 is a detail view of the present invention taken from circle D of FIG. 9.

FIG. 11 is a detail view of the present invention taken from circle E of FIG. 10.

### DETAIL DESCRIPTIONS OF THE INVENTION

All illustrations of the drawings are for the purpose of describing selected versions of the present invention and are not intended to limit the scope of the present invention.

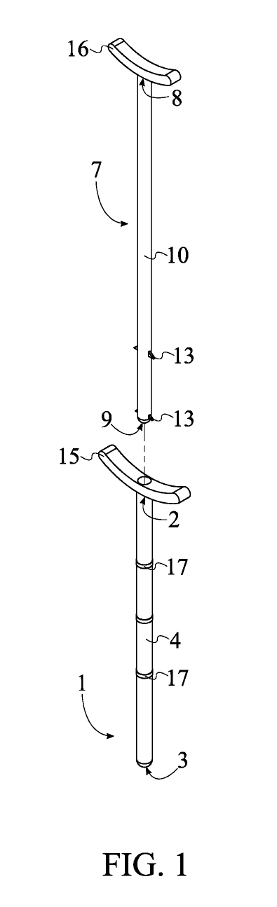

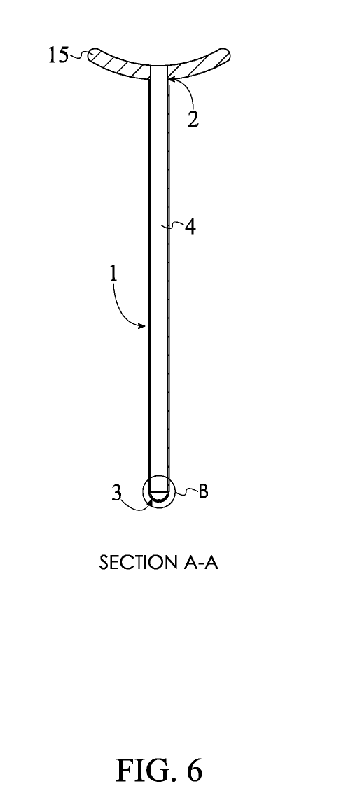

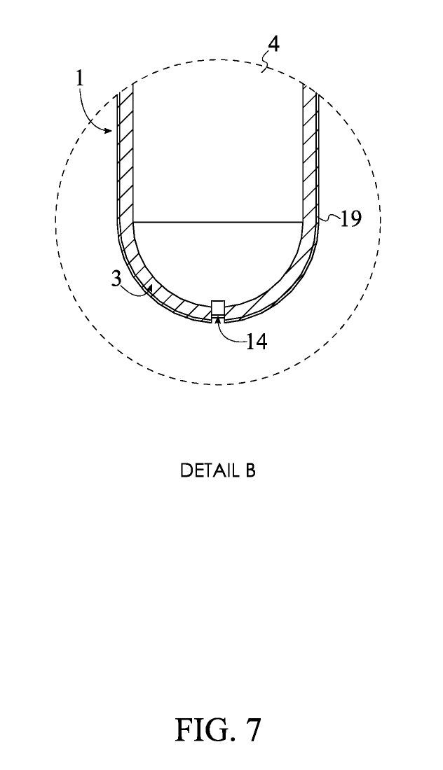

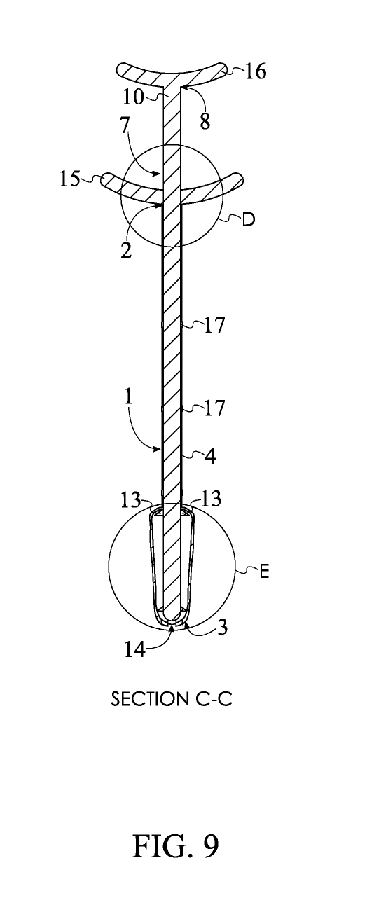

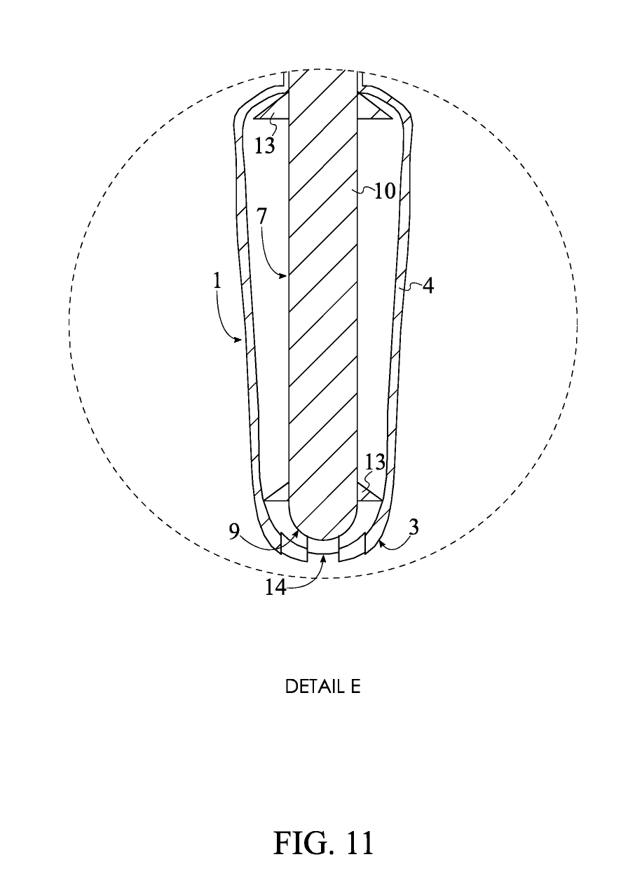

The present invention is a suppository insertion tool that is utilized to administer a suppository within a body orifice. The present invention is additionally able to be inserted into the bowels in order to detect the presence of an obstruction. The present invention is shown in FIGS. 1-11 and comprises a tubular semi-rigid sleeve member 1, a semi-rigid plunger member 7, at least one expansion tab 13, and an expandable opening 14.

The semi-rigid plunger member 7 may be inserted into the tubular semi-rigid sleeve member 1, enabling the tubular semi-rigid sleeve member 1 to serve as an outer casing for the semi-rigid plunger member 7. Because the present invention is designed for internal use within the user's body, in the preferred embodiment of the present invention, the tubular semi-rigid sleeve member 1 and the semi-rigid plunger member 7 are composed of a pliable and hypoallergenic material such as, but not limited to, latex. The tubular semi-rigid sleeve member 1 and the semi-rigid plunger member 7 are sufficiently rigid in order to facilitate insertion into a body orifice such as the rectum.

The tubular semi-rigid sleeve member 1 comprises an insertion open end 2, a suppository-receiving flexible end 3, and a sleeve tubular body 4. The insertion open end 2 is the end into which the semi-rigid plunger member 7 may be inserted. The suppository-receiving flexible end 3 is positioned opposite to the insertion open end 2 and is able to expand in order to accommodate a suppository prior to administration within the body. The sleeve tubular body 4 is the elongated portion of the tubular semi-rigid sleeve member 1 that is able to accommodate the semi-rigid plunger member 7.

The semi-rigid plunger member 7 comprises a grasping end 8, a plunger end 9, and a plunger body 10. The grasping end 8 is the end that is held by the user when inserting the semi-rigid plunger member 7 into the tubular semi-rigid sleeve member 1. The plunger end 9 is positioned opposite to the grasping end 8 and is the end that is inserted into the insertion open end 2. The plunger body 10 is the elongated portion of the semi-rigid plunger member 7 that is inserted into the tubular semi-rigid sleeve member 1.

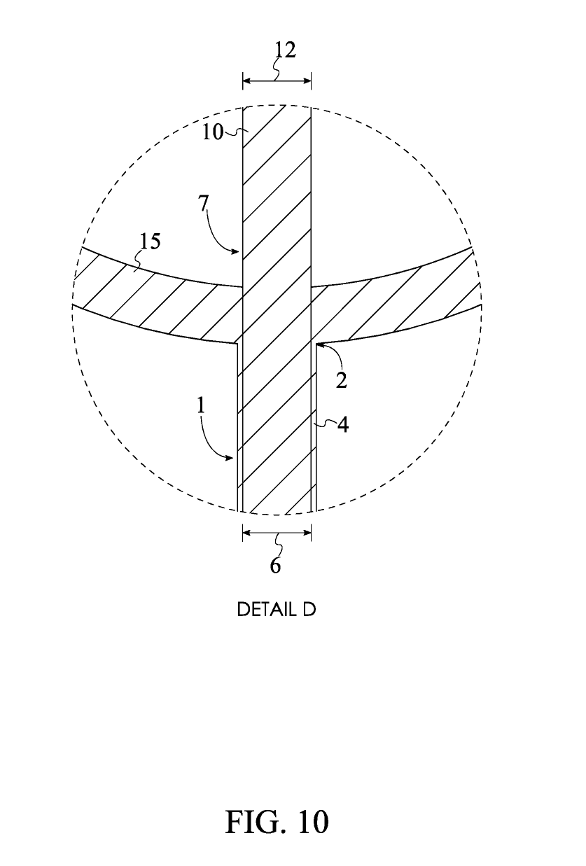

The plunger body 10 is telescopically engaged into the sleeve tubular body 4 at the insertion open end 2. The semi-rigid plunger member 7 is thus able to slide within the tubular semi-rigid sleeve member 1. With reference to FIG. 10, an internal diameter 6 of the sleeve tubular body 4 is equivalent to an external diameter 12 of the plunger body 10. This enables the semi-rigid plunger member 7 and the tubular semi-rigid sleeve member 1 to form an airtight or near-airtight seal when the semi-rigid plunger member 7 is engaged into the tubular semi-rigid sleeve member 1.

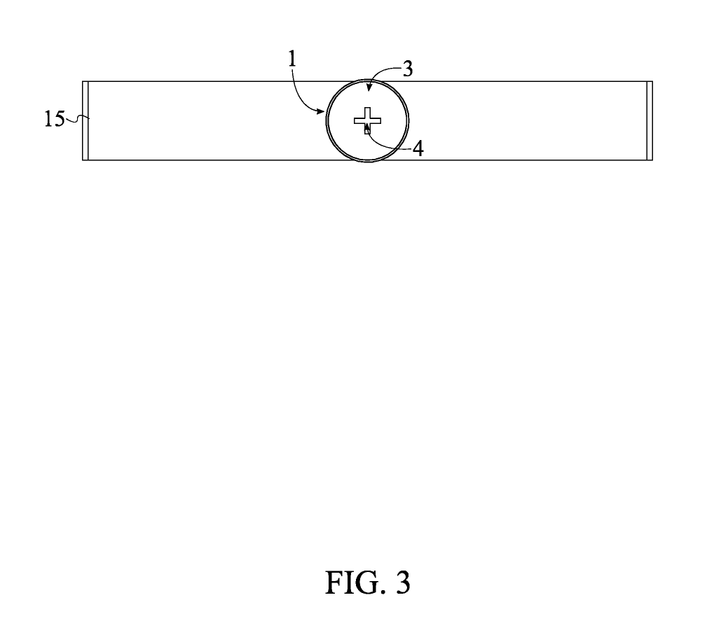

The at least one expansion tab 13 is utilized in order to physically expand the suppository-receiving flexible end 3 as well as the expandable opening 14. The expandable opening 14 longitudinally traverses into the suppository-receiving flexible end 3 and is able to physically expand in size when the semi-rigid plunger member 7 is inserted into the tubular semi-rigid sleeve member 1. A suppository may then be inserted into the suppository-receiving flexible end 3 through the expandable opening 14. The at least one expansion tab 13 is laterally positioned on the plunger body 10, adjacent to the plunger end 9. As such, when the plunger end 9 approaches the suppository-receiving flexible end 3, the at least one expansion tab 13 pushes against the interior of the sleeve tubular body 4, expanding the suppository-receiving flexible end 3 and the expandable opening 14. In the preferred embodiment of the present invention, the expandable opening 14 is cross-shaped as shown in FIG. 3. When the expandable opening 14 is cross-shaped and is expanded, the expandable opening 14 forms a square-like opening.

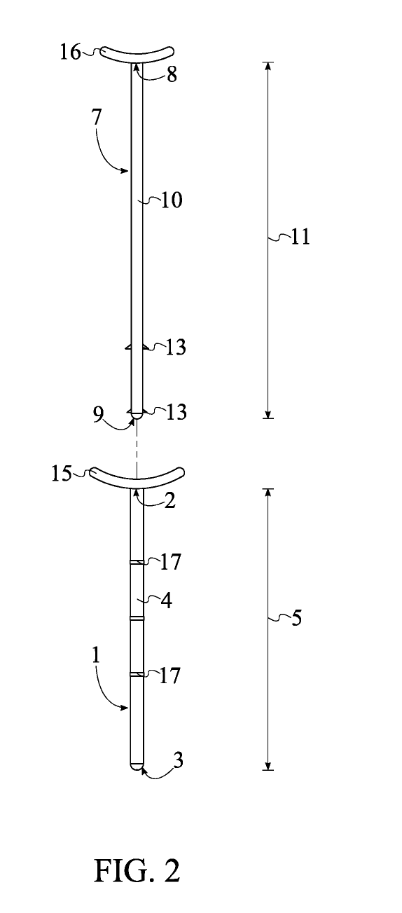

The present invention further comprises a ported sleeve handle 15 and a plunger handle 16. Because the present invention must be inserted into a body orifice during use, the ported sleeve handle 15 and the plunger handle 16 increase the ergonomics of the present invention by facilitating grasping and maneuvering of the tubular semi-rigid sleeve member 1 and the semi-rigid plunger member 7, respectively. The ported sleeve handle 15 is connected to the insertion open end 2 and may be grasped when the tubular semi-rigid sleeve member 1 is inserted into the body orifice. The ported sleeve handle 15 does not impede the progress of the semi-rigid plunger member 7 into the tubular semi-rigid sleeve member 1. The plunger handle 16 is connected to the grasping end 8 and may be grasped when the semi-rigid plunger member 7 is engaged into the tubular semi-rigid sleeve member 1. As shown in FIG. 2, a length 11 of the plunger body 10 is greater than a length 5 of the sleeve tubular body 4. This prevents the semi-rigid plunger member 7 from fully entering the tubular semi-rigid sleeve member 1, enabling a portion of the plunger body 10 as well as the plunger handle 16 to protrude from within the sleeve tubular body 4 when the semi-rigid plunger member 7 is inserted into the tubular semi-rigid sleeve member 1.

In addition to use when inserting a suppository into the body, the present invention may additionally be utilized to detect the presence of an obstruction within the bowels. The present invention further comprises a plurality of color-coded blockage indicators 17 that is able to detect the presence of and the severity of an obstruction within the bowels. For example, the color red may indicate a severe blockage, the color yellow may indicate a moderate blockage, and the color green may indicate a normal condition/no blockage. The plurality of color-coded blockage indicators 17 is laterally distributed along the sleeve tubular body 4 from the suppository-receiving flexible end 3. The plurality of color-coded blockage indicators 17 is thus placed in an easily visible position on the exterior of the sleeve tubular body 4. The plurality of color-coded blockage indicators 17 is offset from each other as well, enabling each of the plurality of color-coded blockage indicators 17 to serve as an indicator for a different depth of obstruction severity within the bowels. The spacing between the plurality of color-coded blockage indicators 17 may be equidistant or of varying lengths.

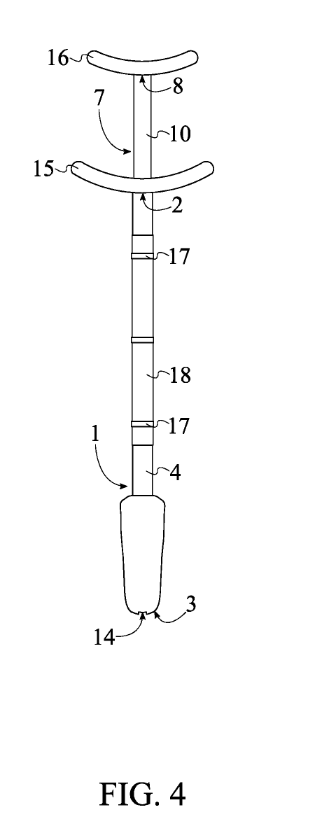

In the embodiment of the present invention shown in FIG. 1 and FIG. 2, the plurality of color-coded blockage indicators 17 is positioned directly on the tubular semi-rigid sleeve member 1. In the embodiment of the present invention shown in FIG. 4, the present invention further comprises a blockage detection sleeve 18. The blockage detection sleeve 18 is a sleeve that may be slid onto and removed from the tubular semi-rigid sleeve member 1 as needed. The blockage detection sleeve 18 is slidably engaged to the sleeve tubular body 4, in between the ported sleeve handle 15 and the suppository-receiving flexible end 3. The blockage detection sleeve 18 is thus able to function in a similar manner as the plurality of color-coded blockage indicators 17 when the plurality of color-coded blockage indicators 17 is positioned directly on the tubular semi-rigid sleeve member 1. In this embodiment of the present invention, the plurality of color-coded blockage indicators 17 is laterally distributed along the blockage detection sleeve 18, for convenient visibility on the blockage detection sleeve 18. The plurality of color-coded blockage indicators 17 is offset from each other as well, allowing each of the plurality of color-coded blockage indicators 17 to serve as an indicator for a different depth of obstruction severity.

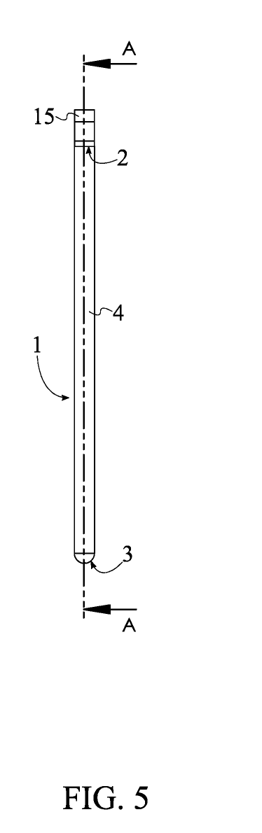

The embodiment of the present invention in FIGS. 5-7 further comprises a chemical reactant coating 19. The chemical reactant coating 19 may be utilized for various applications. One example application is to detect the presence of abnormal pathogens. The chemical reactant coating 19 may serve as an indicator for the presence of abnormal pathogens such as by changing color upon coming into contact with the pathogens. The sleeve tubular body 4 is enveloped by the chemical reactant coating 19 in order to enable the chemical reactant coating 19 to come into contact with the interior of the body orifice. Another example application for the chemical reactant coating 19 is a thermochromic reactant that is able to change color based on exposure to body heat. In this case, the chemical reactant coating 19 may serve as an indicator for the length of time that the present invention has been within the body.

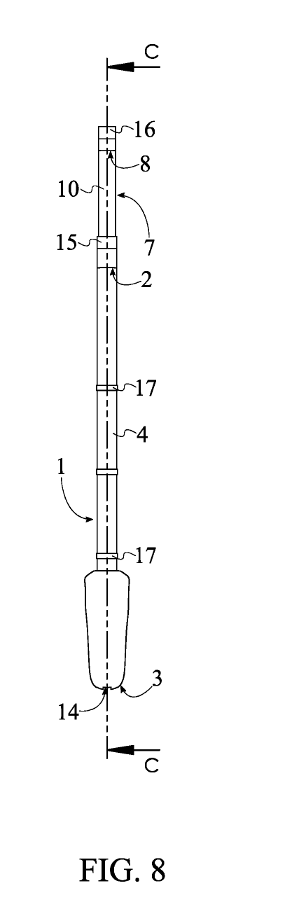

The sleeve tubular body 4 and the expandable opening 14 are shown in an expanded configuration in FIGS. 8-11. The expanded configuration is the configuration in which the expandable opening 14 is expanded after the semi-rigid plunger member 7 is engaged into the tubular semi-rigid sleeve member 1. In the expanded configuration, the plunger end 9 is positioned adjacent to the expandable opening 14. As a result, the at least one expansion tab 13 is positioned adjacent to the expandable opening 14 within the tubular semi-rigid sleeve member 1. The at least one expansion tab 13 is internally pressed against the sleeve tubular body 4, adjacent to the expandable opening 14. This allows the suppository-receiving flexible end 3 and the expandable opening 14 to be expanded. A suppository may then be inserted through the expandable opening 14.

In addition to holding and administering a suppository, the present invention may be utilized to acquire a stool sample from within the bowels. In order to acquire a stool sample, the semi-rigid plunger member 7 is engaged into the tubular semi-rigid sleeve member 1. The tubular semi-rigid sleeve member 1 and the semi-rigid plunger member 7 are then inserted into the rectum. The semi-rigid plunger member 7 may then be drawn outward from the tubular semi-rigid sleeve member 1, generating a vacuum effect and drawing a stool sample into the suppository-receiving flexible end 3.

Although the present invention has been explained in relation to its preferred embodiment, it is understood that many other possible modifications and variations can be made without departing from the spirit and scope of the present invention as hereinafter claimed.

What is claimed is:

1. A suppository insertion tool comprises:
a tubular semi-rigid sleeve member;
a semi-rigid plunger member;
at least one expansion tab;
an expandable opening;
the tubular semi-rigid sleeve member comprises an insertion open end, a suppository-receiving flexible end, and a sleeve tubular body;
the semi-rigid plunger member comprises a grasping end, a plunger end, and a plunger body;
the plunger body being telescopically engaged into the sleeve tubular body at the insertion open end;
the at least one expansion tab being laterally positioned on the plunger body, adjacent to the plunger end; and
the expandable opening longitudinally traversing into the suppository-receiving flexible end.

2. The suppository insertion tool as claimed in claim 1, wherein the expandable opening is cross-shaped.

3. The suppository insertion tool as claimed in claim 1 further comprises:
a ported sleeve handle;
a plunger handle;
the ported sleeve handle being connected to the insertion open end; and
the plunger handle being connected to the grasping end.

4. The suppository insertion tool as claimed in claim 1 further comprises:
a length of the plunger body being greater than a length of the sleeve tubular body.

5. The suppository insertion tool as claimed in claim 1 further comprises:
a plurality of color-coded blockage indicators;
the plurality of color-coded blockage indicators being laterally distributed along the sleeve tubular body from the suppository-receiving flexible end; and
the plurality of color-coded blockage indicators being offset from each other.

6. The suppository insertion tool as claimed in claim 1 further comprises:
a blockage detection sleeve;
a ported sleeve handle;
the ported sleeve handle being connected to the insertion open end; and
the blockage detection sleeve being slidably engaged to the sleeve tubular body, in between the ported sleeve handle and the suppository-receiving flexible end.

7. The suppository insertion tool as claimed in claim 6 further comprises:
a plurality of color-coded blockage indicators;
the plurality of color-coded blockage indicators being laterally distributed along the blockage detection sleeve; and
the plurality of color-coded blockage indicators being offset from each other.

8. The suppository insertion tool as claimed in claim 1 further comprises:
a chemical reactant coating; and
the sleeve tubular body being enveloped by the chemical reactant coating.

9. The suppository insertion tool as claimed in claim 1, wherein an internal diameter of the sleeve tubular body is equivalent to an external diameter of the plunger body.

10. The suppository insertion tool as claimed in claim 1 further comprises:
wherein the sleeve tubular body and the expandable opening are in an expanded configuration;
the plunger end being positioned adjacent to the expandable opening; and
the at least one expansion tab being internally pressed against the sleeve tubular body, adjacent to the expandable opening.

11. A suppository insertion tool comprises:
a tubular semi-rigid sleeve member;
a semi-rigid plunger member;
at least one expansion tab;
an expandable opening;
a plurality of color-coded blockage indicators;
the tubular semi-rigid sleeve member comprises an insertion open end, a suppository-receiving flexible end, and a sleeve tubular body;
the semi-rigid plunger member comprises a grasping end, a plunger end, and a plunger body;
the plunger body being telescopically engaged into the sleeve tubular body at the insertion open end;
the at least one expansion tab being laterally positioned on the plunger body, adjacent to the plunger end;
the expandable opening longitudinally traversing into the suppository-receiving flexible end;
the plurality of color-coded blockage indicators being laterally distributed along the sleeve tubular body from the suppository-receiving flexible end; and
the plurality of color-coded blockage indicators being offset from each other.

12. The suppository insertion tool as claimed in claim 11, wherein the expandable opening is cross-shaped.

13. The suppository insertion tool as claimed in claim 11 further comprises:
a ported sleeve handle;
a plunger handle;
the ported sleeve handle being connected to the insertion open end; and
the plunger handle being connected to the grasping end.

14. The suppository insertion tool as claimed in claim 11 further comprises:
a length of the plunger body being greater than a length of the sleeve tubular body.

15. The suppository insertion tool as claimed in claim 11 further comprises:
a blockage detection sleeve;
a ported sleeve handle;
the ported sleeve handle being connected to the insertion open end; and
the blockage detection sleeve being slidably engaged to the sleeve tubular body, in between the ported sleeve handle and the suppository-receiving flexible end.

16. The suppository insertion tool as claimed in claim 15 further comprises:
the plurality of color-coded blockage indicators being laterally distributed along the blockage detection sleeve; and
the plurality of color-coded blockage indicators being offset from each other.

17. The suppository insertion tool as claimed in claim 11 further comprises:
a chemical reactant coating; and
the sleeve tubular body being enveloped by the chemical reactant coating.

18. The suppository insertion tool as claimed in claim 11, wherein an internal diameter of the sleeve tubular body is equivalent to an external diameter of the plunger body.

19. The suppository insertion tool as claimed in claim 11 further comprises:
wherein the sleeve tubular body and the expandable opening are in an expanded configuration;
the plunger end being positioned adjacent to the expandable opening; and
the at least one expansion tab being internally pressed against the sleeve tubular body, adjacent to the expandable opening.
